(19) United States
(12) Patent Application Publication
(10) Pub. No.: US 2024/0131229 A1
(43) Pub. Date: Apr. 25, 2024

(54) SHAPE MEMORY ALLOY

(71) Applicant: NewSouth Innovations Pty Limited, New South Wales (AU)

(72) Inventor: Xiaopeng Li, Sydney (AU)

(21) Appl. No.: 18/548,286

(22) PCT Filed: Mar. 11, 2022
(86) PCT No.: PCT/AU2022/050203

§ 371 Date: Aug. 28, 2023

(30) Foreign Application Priority Data
Mar. 11, 2021 (AU) 2021900699

(51) Int. Cl. A61L 27/58; A61F 2/30; A61L 27/04; A61L 27/42; B22F 10/28; B22F 10/34; B22F 10/64; B33Y 10/00; B33Y 40/20; B33Y 70/00; B33Y 80/00; C22C 33/02; C22C 38/02; C22C 38/04

(52) U.S. Cl. CPC A61L 27/58; A61F 2/3094; A61L 27/042; A61L 27/047; A61L 27/427; B22F 10/28; B22F 10/34; B22F 10/64; B33Y 10/00; B33Y 40/20; B33Y 70/00; B33Y 80/00; C22C 33/0278; C22C 38/02; C22C 38/04; A61F 2002/3097; A61F 2002/30985; A61F 2310/00017; A61F 2310/00065; A61L 2400/16; A61L 2430/02; B22F 2301/35; B22F 2998/10; B22F 2999/00; C22C 2202/00

(57) ABSTRACT

The present invention is directed to a shape memory alloy, particularly a Fe-based shape memory alloy, that is suitable for use in additive manufacturing methods, as well as methods of use and methods of altering the composition of the alloy during fabrication.

### TECHNICAL FIELD

The present invention relates to shape memory alloys. In particular, the present invention relates to shape memory alloys that are biodegradable. More particularly, the present invention relates to shape memory alloys that are suitable for use in additive manufacturing and that are biodegradable. The invention also relates to methods of manufacturing articles comprising a shape memory alloy and methods for tuning the elemental composition of an alloy. In some forms of the invention, biodegradable shape memory alloys are used to form implantable materials or devices for use in medical treatment. However, it will be appreciated that the invention is not limited to these particular fields of use.

### BACKGROUND ART

The following discussion of the prior art is provided to place the invention in an appropriate technical context and enable the advantages of it to be more fully understood. It should be appreciated, however, that any discussion of the prior art throughout the specification should not be considered as an express or implied admission that such prior art is widely known or forms part of the common general knowledge in the field.

Shape memory materials have the ability to “remember” and recover their original shape after a significant and seemingly plastic deformation or after cooling then heating or other external stimuli. The first known shape memory materials were shape-memory alloys, specifically copper-aluminium-nickel and nickel-titanium (NiTi). Although such alloys are still in use, particularly in engineering applications, such as aeronautical, automotive and robotic actuators, they tend to be expensive given the cost of the elements and careful manufacturing required.

More recently, shape memory alloys based on iron (Fe-SMAs) have been developed. Fe-SMAs are cheaper than copper-aluminium-nickel and nickel-titanium alloys, however they tend to be less stable, are more easily degraded and have generally lower strain recovery than Ni-based SMAs.

Although degradation is generally undesirable for engineering purposes, there are some circumstances where degradation may be desirable. For instance, biodegradable materials are generally absorbed, hydrolysed or otherwise degraded within the body when allowed to do so. This means that materials may perform their function in a body and then be absorbed within the body. Biodegradable materials are of particular relevance as implantable materials for implanting in a human or animal body. Biodegradable medical devices are utilised in circumstances where removing the device is undesirable or difficult (for instance, to promote bone growth across a bone break).

Currently, the most widely-studied biodegradable materials are limited to thermoplastic polymers. Although the introduction of these biodegradable thermoplastic polymers has substantially improved the global healthcare within the last decade, their intrinsic lower strength in comparison with metals and alloys greatly hinders their further penetration in biomedical industries, especially for load-bearing applications. Although Mg-based biodegradable alloys have been developed that address some of the deficiencies in the polymer-based materials, these materials are not suitable as they release hydrogen gas when degrading, which limits the use of these materials to only small devices or specific biological applications. Additionally, most alloys possess a high elastic modulus, which further limits their use in biological applications. For instance, bone has a low modulus of elasticity, and coupling a metal implant with bone can induce severe stress shielding problems, which are undesirable for load bearing biomedical applications.

It is an object of the present invention to overcome or ameliorate at least one of the disadvantages of the prior art, or to provide a useful alternative.

An object of at least one preferred form of the invention is to provide a biodegradable material that is suitable for producing products with shape memory capacity. A further object of at least one preferred form of the invention is to provide a material that has a tuneable elastic modulus and/or other mechanical properties and/or functional properties that could be altered depending on the desired application. A further object of at least one preferred form of the invention is to provide a material that has a biodegradation profile and/or corrosion behaviour that that is tuneable depending on the desired application. Yet a further object of at least one preferred form of the invention is to provide a material that has shape memory abilities.

### SUMMARY

The inventor has developed an alloy comprising Fe, Mn and Si, which has shape memory capabilities and can be used to produce biodegradable devices and products. Further, the inventor has advantageously found that this alloy is suitable for use in metal-based additive manufacturing processes. Furthermore, the inventor has surprisingly found that, when this alloy is used in a laser-based additive manufacturing process, the properties of the fabricated product, such as, for example, the elemental composition, the elastic modulus, the biodegradability, and the strain recovery can be tuned by altering the processing parameters of the additive manufacturing process, allowing a range of products with different properties, for use in a range of applications, to be fabricated from the same starting metal mixture. These properties can be further tuned by the application of optional post-processing steps.

Biodegradable shape memory alloy compositions, methods of manufacture, and devices composed of the biodegradable shape memory alloy compositions are disclosed herein. The production of the alloy in a powder form or in wire or filament form is also disclosed along with manufacture of articles comprising the alloy and using additive manufacturing techniques. The compositions may comprise metallic compositions including, for example, Fe-based alloys. Shape memory biodegradable materials may be used as implantable devices particularly in orthopaedic and spinal surgeries. The materials may also be used in robotics and aerospace applications.

Disclosed herein is a biodegradable shape memory alloy. In some preferred forms of the invention, the biodegradable shape memory alloy is in the form of a metal alloy. Preferably the biodegradable shape memory alloy comprises Fe, Mn and Si. Preferably the biodegradable shape memory alloy is a Fe—Mn—Si alloy. In some forms of the invention, the biodegradable shape memory alloy may have a composition of between 5 and 75% by weight of Mn, between 1 and 15% by weight of Si and the balance Fe. In some preferred forms of the invention, the biodegradable shape memory alloy comprises between 20 and 30% by weight of Mn, between 3 and 9% by weight of Si and the balance Fe. In some preferred forms of the invention, the biodegradable shape memory alloy comprises between 28 and 30% by weight of Mn, between 5 and 7% by weight of Si between 63% and 67% by weight Fe. In some preferred forms of the invention, the biodegradable shape memory alloy consists of Mn at 30% by weight, Si at 6% by weight, and Fe at 64%. In some forms, the biodegradable shape memory alloy may incorporate another element. In some forms, the alloy may further comprise at least one additional element selected from Cr, Ni, Ag, Nb, Ti, V, Co, Al, W, Sn, B, N, C, S and rare earth elements. As the person skilled in the art will appreciate, the alloy may also include unavoidable impurities.

In one aspect of the present invention, there is provided a material for additive manufacturing, the material comprising: Mn at between 25 and 35% by weight; Si at between 5 and 8% by weight; Fe at between 60 and 70% by weight, and optionally comprising at least one additional element selected from Cr, Ni, Ag, Nb, Ti, V, Co, Al, W, Sn, B, N, C, S and rare earth elements. In some preferred forms of the invention, the material is a powder. The powder may be blended from elemental powders, or is prealloyed and then ground/comminuted to form a powder.

In some forms of the invention, disclosed herein is a biodegradable shape memory alloy device formed using additive manufacturing. In some forms disclosed herein the biodegradable shape memory alloy is formed from alternative manufacturing techniques. In some forms of the invention there is provided an alloy powder composed of an alloy for additive manufacturing. In some forms of the invention disclosed herein there is provided a filament or wire composed of an alloy for additive manufacturing. In some forms of the invention the alloy is a Fe—Mn—Si alloy.

Also disclosed herein is a process for manufacturing an article, comprising: obtaining the alloy of the invention or material described above (i.e., powder, wire or filament) for use in an additive manufacturing process; and carrying out the additive manufacturing process to obtain the article. It will be appreciated that the article recovers from strain with heating and/or other external stimuli. In some forms of the invention, the recovered strain is about 10%. In some forms of the invention, the recovered strain is about 8%. In some forms of the invention, the recovered strain is about 4%. In some forms of the invention, the recovered strain is between 0.5 to 1, 1 to 1.5, 1.5 to 2, 2 to 2.5, 2.5 to 3, 3 to 3.5, 3.5 to 4, 4 to 4.5, 4.5 to 5, 5 to 5.5, 5.5 to 6, 6 to 6.5, 6.5 to 7, 7 to 7.5, 7.5 to 8, 8 to 8.5, 8.5 to 9, 9 to 9.5, 9.5 to 10, 10 to 10.5, 10.5 to 11, 11 to 11.5, 11.5 to 12, 12 to 12.5, 12.5 to 13, 13 to 13.5, 13.5 to 14, 14 to 14.5, 14.5 to 15, 15 to 15.5, 15.5 to 16, 16 to 16.5, 16.5 to 17, 17 to 17.5, 17.5 to 18, 18 to 18.5, 18.5 to 19, 19 to 19.5, or 19.5 to 20%, for example, 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, or 20%.

In some forms of the invention, the strain is recovered upon heating. In some forms of the invention, the heating comprises heating the article to at least room temperature, or at least 30° C., or at least 40° C., or at least 50° C., or at least 60° C., or at least 70° C., or at least 80° C., or at least 90° C., or at least 100° C., or at least 150° C., or at least 200° C., or at least 300° C., or at least 400° C., or at least 500° C., least 600° C., or at least 700° C., or at least 800° C., or at least 900° C., or at least 1000° C., or at least 1100° C. or at least 1200° C.

In some forms of the invention, the heating comprises heating the article to about 20, 25, 30, 35, 40, 45, 50, 55, 60, 70, 80, 100, 120, 140, 160, 180, 200, 220, 240, 260, 280, 300, 320, 340, 360, 380, 400, 420, 440, 460, 480, 500, 520, 540, 560, 580, 600, 620, 640, 660, 680, 700, 720, 740, 760, 780, 800, 820, 840, 860, 880, 900, 920, 940, 960, 980, 1000, 1020, 1040, 1060, 1080, 1100, 1120, 1140, 1160, 1180, or 1200° C., for example, between about 20 to 40, 40 to 60, 60 to 80, 80 to 100, 100 to 120, 120 to 140, 140 to 160, 160 to 180, 180 to 200, 200 to 220, 220 to 240, 240 to 260, 260 to 280, 280 to 300, 300 to 320, 320 to 340, 340 to 360, 360 to 380, 380 to 400, 400 to 420, 420 to 440, 440 to 460, 460 to 480, 480 to 500, 500 to 520, 520 to 540, 540 to 560, 560 to 580, 580 to 600, 600 to 620, 620 to 640, 640 to 660, 660 to 680, 680 to 700, 700 to 720, 720 to 740, 740 to 760, 760 to 780, 780 to 800, 800 to 820, 820 to 840, 840 to 860, 860 to 880, 880 to 900, 900 to 920, 920 to 940, 940 to 960, 960 to 980, 980 to 1000, 1000 to 1020, 1020 to 1040, 1040 to 1060, 1060 to 1080, 1080 to 1100, 1100 to 1120, 1120 to 1140, 1140 to 1160, 1160 to 1180, or 1180 to 1200° C.

In some forms of the invention, the additive manufacturing process is a laser powder bed fusion (PBF) process, or a selective laser melting (SLM) process, or an electron beam melting (EBM) process, or material extrusion, or binder jetting, or material jetting. Other equivalent or similar methods will be known to the person skilled in the art.

In some forms of the invention, the article comprises a medical device, particularly an implantable medical device. In some forms of the invention, the article is a bone implant. Other implantable medical devices will be known to the person skilled in the art.

In some forms of the invention, the process further comprises at least one post-processing step. Preferably the post-processing step is a homogenization step or a stress relief step or a heat treatment step with or without pressure or any combination thereof. The at least one post-processing step may comprise heating the article to at least 300° C., or at least 400° C., or at least 500° C., or at least 600° C., or at least 700° C., or at least 800° C., or at least 900° C., or at least 1000° C., or at least 1100° C., or at least 1200° C., or at least 1300° C., or between about 200° C. and about 1500° C., or between 500° C. and 1000° C., or between 350° C. and 850° C., or between 750° C. and 1250° C., or between 1000° C. and 1200° C., or to about 200, 250, 300, 350, 400, 450, 500, 550, 600, 650, 700, 750, 800, 850, 900, 950, 1000, 1050, 1100, 1150, 1200, 1250, 1300, 1350, 1400, 1450 or 1500° C. or any range therein. Once heated, the homogenization step may comprise holding the article at that temperature for at least 30 minutes, or at least 1 hour, or at least 2 hours, or at least 3 hours, or at least 4 hours, or at least 5 hours, or at least 6 hours, or at least 7 hours, or at least 8 hours, or at least 9 hours, or at least 12 hours, or at least 15 hours, or at least 18 hours, or at least 21 hours, or at least 24 hours, or at least 30 hours, or at least 36 hours, or at least 48 hours, or at least 72 hours, or between about 30 minutes and 24 hours, or between 1 and 90 hours, or between 2 and 72 hours, or between 5 and 60 hours, or between 10 and 36 hours, or between 12 and 24 hours, or between 5 and 65 hours, or between 15 and 55 hours, or for about 30 minutes, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89 or 90 hours or any range therein.

In some forms of the invention, the article is relatively more biodegradable than the alloy.

Also disclosed herein is a process for tuning the elemental composition of an alloy, comprising the steps of: obtaining an alloy comprising at least two elements for use in an additive manufacturing process; and exposing the alloy to an additive manufacturing process; whereby, in use, at least a portion of at least one element of the alloy is evaporated during the additive manufacturing process. It will be appreciated that the process can be used to selectively evaporate one or more elements in order to achieve a predetermined final alloy composition. In some forms of the invention, the at least one element of the alloy that is evaporated has the lowest melting point temperature. In some forms of the invention, the alloy is as described above. In some forms of the invention, the element that is at least partially evaporated is Mn and/or Si. In some forms of the invention, the evaporation is controlled by adjusting the parameters of the additive manufacturing process, as will be discussed further below.

### Definitions

In describing and claiming the present invention, the following terminology will be used in accordance with the definitions set out below. It is also to be understood that the terminology used herein is for the purpose of describing particular embodiments of the invention only and is not intended to be limiting. Unless defined otherwise, all technical and scientific terms used herein have the same meaning as commonly understood by one having ordinary skill in the art to which the invention pertains.

Unless the context clearly requires otherwise, throughout the description and the claims, the terms “comprise”, “comprising”, and the like are to be construed in an inclusive sense as opposed to an exclusive or exhaustive sense; that is to say, in the sense of “including, but not limited to”. For example, a composition, mixture, process or method that comprises a list of elements is not necessarily limited to only those elements but may include other elements not expressly listed or inherent to such composition, mixture, process or method.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition, process or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Where applicants have defined an invention or a portion thereof with an open-ended term such as “comprising”, it should be readily understood that (unless otherwise stated) the description should be interpreted to also describe such an invention using the terms “consisting essentially of” or “consisting of.” In other words, with respect to the terms “comprising”, “consisting of”, and “consisting essentially of”, where one of these three terms is used herein, the presently disclosed and claimed subject matter may include the use of either of the other two terms. Thus, in some embodiments not otherwise explicitly recited, any instance of “comprising” may be replaced by “consisting of” or, alternatively, by “consisting essentially of”.

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be non-restrictive regarding the number of instances (i.e., occurrences) of the element or component. Therefore “a” or “an” should be read to include one or at least one, and the singular word form of the element or component also includes the plural unless the number is obviously meant to be singular.

The examples are not intended to limit the scope of the invention. In what follows, or where otherwise indicated, “%” will mean “weight %”, “ratio” will mean “weight ratio” and “parts” will mean “weight parts”.

The terms “predominantly” and “substantially” as used herein shall mean comprising more than 50% by weight, unless otherwise indicated.

The recitation of a numerical range using endpoints includes all numbers subsumed within that range (e.g., 1 to 5 includes 1, 1.5, 2, 2.75, 3, 3.80, 4, 5).

The terms “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The term “iron-based alloy” and variations thereof as used herein refers to shape memory alloys that comprise iron. The term is not intended to require that iron is “predominantly” or “substantially” present, only that it is present in some amount.

The term “shape memory alloy” as used herein refers to an alloy that can be deformed when cold, but returns to a pre-deformed (pre-determined) shape when heated or exposed to some other external stimuli.

### BRIEF DESCRIPTION OF THE DRAWINGS

FIG. 1 illustrates the distinction between one-way and two-way shape memory.

FIG. 2 illustrates the change in elemental composition of the alloy that is achieved following additive manufacturing, compared to the reference (that is, starting alloy material) in terms of (a) Fe, (b) Mn and (c) Si proportions.

FIG. 3 provides stress-strain curves for samples produced with a laser PBF process followed by post-production homogenization, whereby the laser power and/or speed was varied.

FIG. 4 illustrates the effect of post-production homogenization on articles fabricated by a laser PBF process.

FIG. 5 illustrates the effect on biodegradation of fabricated alloy samples compared to the reference alloy.

FIG. 6 illustrates that the modulus of the fabricated alloy articles can be altered by varying the fabrication parameters.

FIG. 7 illustrates a characteristic biodegradation profile of a useful implantable medical device.

FIG. 8 illustrates the strain recovery of the alloy following additive manufacturing.

FIGS. 9 and 10 illustrate the characteristic transformation temperatures of alloys that are prepared by additive manufacturing processes.

### DETAILED DESCRIPTION OF THE DISCLOSURE

In the following detailed description, reference is made to the accompanying drawings which form a part of the detailed description. The illustrative embodiments described in the detailed description, depicted in the drawings and defined in the claims, are not intended to be limiting. Other embodiments may be utilised, and other changes may be made without departing from the spirit or scope of the subject matter presented. It will be readily understood that the aspects of the present disclosure, as generally described herein and illustrated in the drawings may be arranged, substituted, combined, separated, and designed in a wide variety of different configurations, all of which are contemplated in this disclosure. Hence, the following detailed description does not limit the scope of the present invention, which is defined only by the claims.

The present invention relates to a shape memory alloy, particularly an iron-based shape memory alloy, more particularly an iron-based shape memory alloy that can be used to form useful products using additive manufacturing methods.

In particular, the inventor has advantageously found an iron-based alloy composition that is suitable for use with additive manufacturing techniques. As will be described in more detail below and with reference to the examples, the iron-based alloy composition of the present invention is able to be “trained” as a shape memory alloy and is biodegradable. The inventor has also usefully found that properties such as the elemental composition, microstructure and mechanical properties of the alloy can be tuned, or altered, by adjusting the properties of the additive manufacturing process itself and/or by post processing step(s).

It is anticipated by the inventor that the present invention may lead to the more efficient, and more cost-effective manufacturing of a variety of devices, in particular medical devices, that may be beneficially biodegradable, and have shape memory functions. The cost-effectiveness is expected to be achieved by the use of iron-based alloys (which are cheaper than other shape memory alloys known in the art), the use of additive manufacturing processes to easily produce complex or fine designs, and the ability to produce alloys with a range of compositions from the same starting material. These materials may be used to compose orthopaedic implants, fixations, scaffolds, screws, wires and the like without the need for secondary or removal surgery. The materials may also be used in children who are still growing as the medical devices fabricated through the method may be dissolved or absorbed through the healing process while maintaining functionality as needed.

Alloy

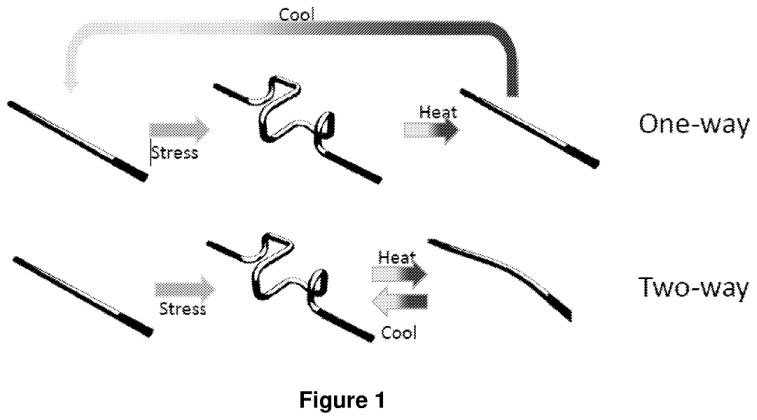

The alloy of the present invention suitably comprises iron (i.e., is an iron-based alloy). The alloy may also comprise at least one element selected from the group consisting of Pt, Pd, Si, Cu, Cr, Mn, Ni, Ag, Nb, Ti, V, Co, Al, W, Sn, B, N, C, S, or rare earth elements. As the person skilled in the art would appreciate, the presence of at least one of these elements would be expected to influence the microstructure of the alloy, such that the solid alloy maintains a martensitic structure at room temperature, transitions to an austenitic structure with heating, and then cools to a martensitic structure, whereby plastic deformation that occurs during the martensitic phase is recovered after transition to the austenitic phase (that is, a shape memory alloy). As shown in FIG. 1, this memory effect can take one of two different types: a one-way memory effect; and a two-way memory effect. Generally, with a one-way memory effect, the material remembers one shape, which is recovered by heating from deformed cold state; no shape transformation occurs when cooling. However, with a two-way memory effect, the material remembers two shapes, a high-temperature shape and a low-temperature shape, and the material can either oscillate between these two shapes with heating and cooling (i.e., intrinsic two-way effect) or following deformation in the cold state (so long as the material is not overheated, which can result in erasure of the low-temperature shape. As the skilled person would appreciate, these effects are a result of “training” of the material, rather than any particular feature of the material (although some shape memory alloys may not be capable of a two-way memory effect). The alloy of the present invention may be capable of exhibiting one-way shape memory effects, or two-way shape memory effects.

The alloy of the present invention may be an alloy comprising, or consisting of, Fe, Cr and Si (i.e., Fe—Cr—Si), or it may comprise, or consist of, Fe and Pt (i.e., Fe—Pt), or it may comprise, or consist of, Fe and Pd (i.e., Fe—Pd), or it may comprise, or consist of, Fe and Cu (i.e., Fe—Cu), or it may comprise, or consist of, Fe and Co (i.e., Fe—Co), or it may comprise, or consist of, Fe and Cr (i.e., Fe—Cr), or it may comprise, or consist of, Fe and Mn (i.e., Fe—Mn) or it may comprise, or consist of, Fe and Ni (i.e., Fe—Ni), or it may be any combination of these. In one particular embodiment, the alloy comprises, or consists of, Mn and Si (i.e., Fe—Mn—Si). The alloy may comprise between 5% and 75% by weight Mn, between 1% and 12% by weight Si, and the balance (excluding impurities) being Fe (that is, between 13% and 94% by weight Fe). The alloy may optionally comprise elements other than Fe, Mn and Si. As the skilled person would appreciate, in such alloys the additional element replaces at least a portion of the Fe. For example, in an alloy comprising 70% Mn, 5% Si and 20% Cr, the Fe is present at 5% by weight.

In particular, the alloy may comprise, or consist of, by weight of the total alloy:
Mn at between 5% and 75%, or between 10% and 70%, or between 15% and 65%, or between 20% and 40%, or between 25% and 50%, or between 20% and 60%, or between 30% and 75%, or between 35% and 70%, or between 40% and 67%, or between 55% and 66%, or between 55% and 70%, or between 60% and 65%, or between 25% and 35%, or at 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74 or 75% by weight or any range therein;
Si at between 1% and 12%, or between 2% and 10%, or between 3% and 9%, or between 5% and 8%, or between 6% and 9%, or between 3% and 6%, or at 1, 1.5, 2, 2.5, 3, 3.5, 4, 4.5, 5, 5.5, 6, 6.5, 7, 7.8 or 8% by weight or any range therein; and
the balance (excluding additional elements and impurities) being Fe, whereby the maximum Fe content is 94% and the minimum Fe content is 5%, such as between 5% and 90%, or between 10% and 50%, or between 15 and 70%, or between 20% and 85%, or between 25% and 80%, or between 30% and 75%, or between 35% and 70%, or between 40% and 65%, or between 45% and 75%, or between 50% and 70%, or between 52% and 67%, or between 55% and 66%, or between 55% and 70%, or between 60% and 65%, or between 25% and 35%, or at, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86 or 87% by weight or any range therein.

In one embodiment of the present invention, there is provided an alloy comprising between 20 and 40% by weight Mn, between 3 and 8% by weight Si and between 52 and 77% by weight Fe. In a particular embodiment of the present invention, there is provided an alloy comprising between 27 and 31% by weight Mn, between 4 and 8% by weight Si and between 62 and 68% by weight Fe (that is, 29% Mn (±2 wt %), 6% Si (±2 wt %), 65% Fe (±2 wt %)).

Additive Manufacturing

As the skilled person would appreciate, additive manufacturing (also known in the art as 3D printing or 4D printing) allows for the formation of specific structures designed for a particular use rather than standardised device sizes and shapes. Additive manufacturing techniques allow for customised and complex shapes to produced, often with fine geometries and with small tolerances, for engineering, aerospace, medical or other devices or uses. Additive manufacturing is referred to as such since it produces articles by adding materials, which differs from conventional manufacturing techniques that are generally subtractive in nature.

It is envisioned that the alloy of the present invention is for use in any suitable additive manufacturing process. In particular, any additive manufacturing process that uses a metal as a substrate would be suitable for use with the alloy of the present invention. Current techniques such as powder bed fusion processes, powder-fed directed-energy deposition processes, extrusion-based processes, metal wire processes, binder jetting or material jetting processes would be suitable for use with the alloy of the present invention. In particular:
powder bed fusion (PBF) processes (which includes selective laser melting (SLM) processes) use a laser or electron beam as an energy source to melt metal powder at the focus point of the laser or electron beam. The PBF process begins with a 3D CAD model, which is converted into discrete layers. For each layer, a heat source path is plotted and the laser or electron beam is then driven along this path across a bed of metal powder, heating and fusing the metal powder together. Once one path is completed, metal powder is then applied to the surface and the process repeated until the manufacture is complete;
powder-fed directed-energy deposition is similar to PBF described above, which also uses a laser or electron beam as an energy source to melt metal powder. However, in this process, the metal powder is supplied through the deposition head that focusses the laser or electron beam, rather than as a bed of metal powder;
extrusion-based processes are hybrid processes, whereby the feedstock is a combination of a polymeric binder and a fine-grain metal powder, with the final article undergoing post-processing steps of debinding and sintering in order to achieve the final metal product. In other words, rather than heating the metal in place (as the above processes do), extrusion-based processes produce a metal-binder hybrid, whereby the binder is then removed, and metal particles heated and fused together; and
metal wire processes, such as Laser Metal Deposition-wire (LMD-w) or electron beam freeform fabrication, are similar to powder-based processes above, but instead the metal substrate is in the form of a wire, rather than a powder, with a heat source (such as an electron beam or a laser) heating and melting the metal wire in place to build up a 3-dimensional product.

Although the above processes are known techniques that would be suitable for use with the alloy of the present invention, it is anticipated that the alloy could be used with any additive manufacturing process that heats and fuses metal(s) together, including processes not yet developed (such as, for example, liquid metal processes). In other words, the present invention is not limited to use with the above techniques. In certain embodiments, the inventor has demonstrated that the alloy of the present invention is suitable for use with a laser-based powder bed diffusion (PBD) technique.

The form of the alloy for use in an additive manufacturing process may be any suitable form. For instance, with reference to the existing processes described above, the alloy may be as a powder, a wire, a filament, or a powder-binder hybrid mixture or any other suitable form. These forms may be produced by any suitable method known in the art. For example, powders of the alloy can be either prepared by mixing and blending elemental powders (for instance, Fe, Mn and Si and other additional elements can be mixed to form a homogenous powder, which are then alloyed when heated by the laser or electron beam) using various powder mixing and blending techniques including, for example, ball milling, mechanical blending using a mixer, or like techniques known to the skilled person. As the skilled person will appreciate, a powder for use in an additive manufacturing process that has been formed by mixing elemental powders together is not, in the strict definition of the term, an “alloy” until it is heated, melted and the elements combined into a solid solution during the manufacturing process. Accordingly, the term “material” is used herein to define a starting material that has not yet been alloyed, but which is capable of producing an alloy of the present invention, particularly when used in a metal-based additive manufacturing process described above.

Alternatively, powders of the alloy can be prepared in a pre-alloyed form from producing pre-alloyed powders via various powder manufacturing techniques including gas atomization, plasma atomization, and the like where pre-alloyed ingots with different compositions can be produced through different melting techniques including arc melting and other standard alloying processes. Similarly, wires and/or filaments of the alloy can be produced in a pre-alloyed form and subsequently drawn out into their usable form, or separate wires or filaments of each element can be drawn and used together, wherein they are alloyed together by the heat source (such as a laser or electron beam). As the skilled person would appreciate, the composition of the alloy can be controlled at the raw material preparation stage (that is, with the mixing of elemental powders, or pre-alloying before milling or drawing into wires or filaments).

As the skilled person would also appreciate, the metal-based additive manufacturing processes described above have a range of processing parameters that may need to be optimised depending on a range of factors, such as the characteristics of the metal substrate, the desired characteristics of the finished product, the characteristics of the particular additive manufacturing device, and the like. It would be anticipated that the skilled person could optimise such parameters when using the alloy of the present invention, depending on the nature of the product being produced. By way of example, in laser powder bed fusion additive manufacturing, the key processing parameters include, but are not limited to, laser power, laser scan speed, hatch spacing, scanning strategy, layer thickness, laser beam shape and spot size, single scan or multiple scans, and the like. The laser system can also be different including the wavelength of the laser (e.g., 1060 nm or 532 nm, and so on), power range, scan speed, single mode or multimode, beam shape, single laser system or multiple laser systems, for instance. The laser powder bed fusion additive manufacturing can also be carried out in different, usually inert, atmospheres including, for example, argon or nitrogen, or other gas atmospheres including mixed gases.

Although the parameters of an additive manufacturing process are generally optimised with a view to the physical or mechanical properties of the finished product, and/or the properties of the substrate material being used, the inventor of the present invention has also found that, at least in metal-based additive manufacturing processes using the alloy of the present invention, the elemental composition of the finished product can be tuned. In other words, under certain conditions, the elemental composition of the finished product will be different from the starting alloy in regard to at least one element. Without being bound to theory, the inventor understands that, during heating of the metal alloy powder or wire, at least a portion of one (or more) elements in the alloy may be “evaporated” and hence removed from the fabricated product, resulting in a relative decrease in the percentage by weight of the “evaporated” element(s), and a relative increase in the percentage by weight of the remaining element(s). In other words, it is an advantage of the present invention that metal products with different elemental compositions can be fabricated from the same starting alloy, leading to manufacturing efficiencies and a decrease in the cost of fabricating a range of products.

Alloy Products

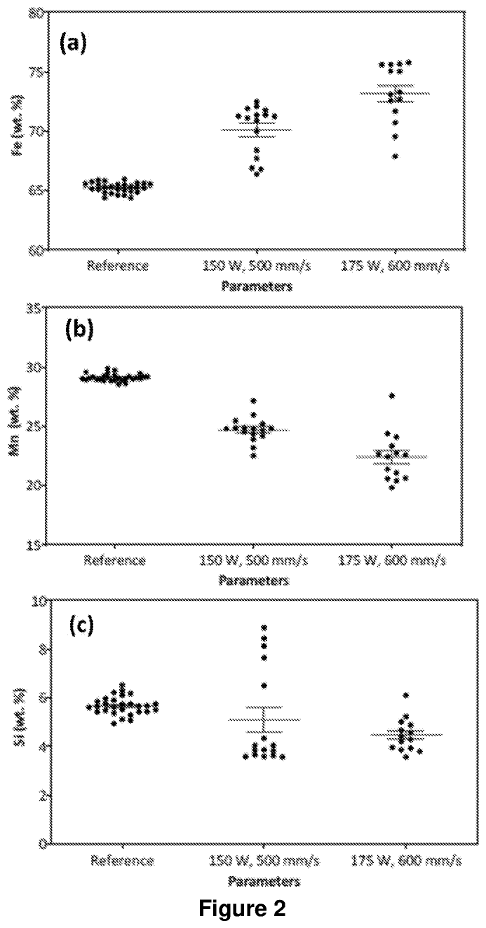

As shown in more detail in the examples below, the inventor has found that parameters of additive manufacturing techniques, such as laser power and laser scanning speed, can be used to alter the elemental composition of the alloy of the present invention when analysed using energy-dispersive x-ray spectroscopy (EDS). For example, in certain embodiments, the inventor has found that a starting alloy of 29% Mn (±2 wt %), 6% Si (±2 wt %), 65% Fe (±2 wt %) can be used to manufacture products that have a composition of between about 19 and 27 wt % Mn, between 3 and 9 wt % Si and between 66 wt % and 77 wt % simply by varying the laser power and laser scanning speed. Further, adjusting the laser power and/or the laser scan speed can result in a tuning of the composition of the final product. By way of an example, the inventor has found that a starting alloy of 29% Mn (±2 wt %), 6% Si (±2 wt %), 65% Fe (±2 wt %) can be used to obtain products with an average composition of 25% Mn (±3 wt %), 5% Si (±4 wt %), 70% Fe (±4 wt %) by using a laser powder bed fusion process at a laser power of 150 W and a scanning speed of 500 mm/s, and products with an average composition of 22% Mn (±4 wt %), 4.5% Si (±2 wt %), 74% Fe (±7 wt %) by using a laser powder bed fusion process at a laser power of 175 W and a scanning speed of 600 mm/s (with all other parameters kept constant) (see FIG. 2). In other words, the inventor has shown generally that, when the alloy of the present invention is heated in a laser powder bed fusion process, the Mn and Si are generally at least partially evaporated (leading to a relative decrease in concentration in the finished product) and the Fe is not, leading to a relative increase in Fe concentration in the finished product. This provides the skilled person with the ability to adapt the composition of the finished product by changing processing conditions. Without being bound to any particular theory, it is understood that the laser used in the powder bed fusion process induces evaporation of at least a portion of the Mn and Si as these elements have lower melting points (Mn=1246° C.; Si=1410° C.) than the Fe (1538° C.). Accordingly, the skilled person can apply this principal generally and anticipate that elements with lower melting points will preferentially evaporate to a greater extent than elements with higher melting points, and element(s) that have the highest melting point would be generally expected to increase in their relative concentration in the finished alloy product. The variation to the relative proportions of each of the elements as a result of evaporation of the lowest melting point element(s) may be at least ±5%, or at least ±10%, or at least ±15%, or at least ±20%, or at least ±25%, or at least ±30%, or at least ±35%, or at least ±40%, or at least ±45%, or at least ±50%, or more.

The inventor has also advantageously found that altering the parameters of the additive manufacturing process can allow for the tuning of other properties of the alloy product. As an example, in data not shown, the inventor has found that the microstructure of alloys that are printed from an alloy of the present invention differs as a function of laser power, with products obtained are a higher laser power comprising larger, more ordered (i.e., parallel) grains, with lower laser powers resulting in microstructures comprising increasingly smaller, more randomly-oriented grains.

Post-Processing

Optionally, once a product is formed using an additive manufacturing technique and comprising an alloy of the present invention, it may be subjected to post-processing techniques. As the skilled person would be aware, products formed from additive manufacturing, and particularly metallic products formed from additive manufacturing, require post-processing steps to relieve stresses that form during manufacturing. Additionally, shape memory alloys generally require “training” so that the product “remembers” the preferred cold-state (and, in the case of two-way shape memory products, the preferred hot-state) shape. The shape memory behaviour of the additive manufactured products comprising an alloy of the present invention can also be enhanced using post-processing techniques.

Accordingly, optional post-processing steps carried out on the manufactured products of the present invention include homogenization, thermomechanical processing (or thermomechanical training), precipitation and hot isostatic pressing.

Homogenization

A common technique for reducing stress caused by defects in metal products, homogenization (also referred to as diffusion or homogenizing annealing) is a process whereby the metal is heated to within 80-90% of its melting temperature and usually held at this elevated temperature for a period of time (which may be up to about 48 hours) to remove micro irregularities that formed during production. Homogenization is known to improve the plasticity of alloys, increases mechanical stability and decreases the directionality of the microstructure.

Thermomechanical Processing/Training

A widely used technique to improve the shape memory behaviour of these alloys is thermomechanical training. This process exposes the alloys to a series of thermal and mechanical stress that suppresses the slip deformation and stimulates the formation of homogenous and thin martensite plates. The training treatment increases the austenite finish temperature and suppresses martensitic temperature during thermal cycling from, for example, 300-350° C. (although the skilled person would be aware that this is only an example and not exclusive for all alloys). However, the skilled person would appreciate that for each alloy, there is an optimum number of training cycle to prevent the decrease in the quantity of the transformed phase. During thermomechanical training, the thermal treatment of alloy is equivalent to homogenization described above (e.g., which can be done at 1,100° C. from 0.5 to 48 hrs or even longer for alloys of the present invention), which improves the internal structure during the training cycles and is achieved by introduction of the certain phases distributed in fine structure which favours the shape memory properties.

Precipitation

Another effect of thermomechanical treatments is the evolution of precipitates that significantly improve the shape memory effect of alloys (in particular, the Fe—Mn—Si-based alloys of the present invention).

It has been observed previously that clusters of fine particles are associated with many stacking faults. The stacking faults were likely artefacts produced during the sample preparation as the result of low stacking energy. The large elastic strain around the precipitates promotes the formation of new stacking faults, growing along an orientation different from the pre-existing one. It is assumed by the inventor that the precipitates, which nucleate on top of stacking faults, can act as nucleation sites of stacking faults with different orientation, the phase transformation would in consequence takes place while the sample underwent high enough stress. The interface energy of the precipitates particle and matrix can then promote the formation of stacking faults and martensite. It is apparent from previous studies that the presence of precipitates induced through the ageing process after pre-stressing (i.e. cold working or tensile loading) can improve the yield strength of the parent austenite (i.e., y) phase and the shape memory effect without the need for thermomechanical training.

Hot Isostatic Pressing (HIPping)

In addition, some other post processing techniques suitable of the alloys of the present invention can enhance the shape memory effect and other properties of the fabricated alloys as well. For example, hot isostatic pressing (HIPping) can help reduce or eliminate the processing defects in the additive manufacturing fabricated alloys. For example, HIPping conditions of 1100° C. and 100 MPa for 3 hours may be suitable for post-processing of the alloy products of the present invention.

As the skilled person would appreciate, different post-processing treatment techniques (as discussed herein or otherwise known to the skilled person) can either be used alone or in any suitable combination to post process the fabricated alloys. For example, the additive manufacturing fabricated alloys of the present invention can be HIPped and then homogenised.

In general, shape memory alloy products, particularly for use in the construction industry, require four steps; shaping, training, pre-strain, and recovery of shape. The thermomechanical processing involves a series of cyclic loading and recovery, thereby increasing the processing cost. However, the inventor of the present invention considers that, by using additive manufacturing techniques to control alloy composition and microstructure, it may be possible to manufacture devices, such as patient-specific bone implants and the like, without requiring costly post-processing procedures. However, the skilled person would also appreciate that, for some uses, post-processing of the additive manufactured products comprising an alloy of the present invention will be unavoidable.

Properties

The inventor has advantageously found that products formed from the alloy of the present invention, using additive manufacturing processes, have a range of properties that may be useful for certain applications. These properties will be discussed in further detail below.

Biodegradability

Although the degradation of metal articles and devices produced by additive manufacturing is generally undesirable (particularly for engineering and aeronautical applications), there are some applications whereby some degradation is useful. One such use is in the development of biological implants that dissolve or breakdown after a period of time. Such transient implants or devices are designed to have a limited lifetime, whereby the implant may be difficult to remove from the patient, and/or harmful if allowed to persist within the patient.

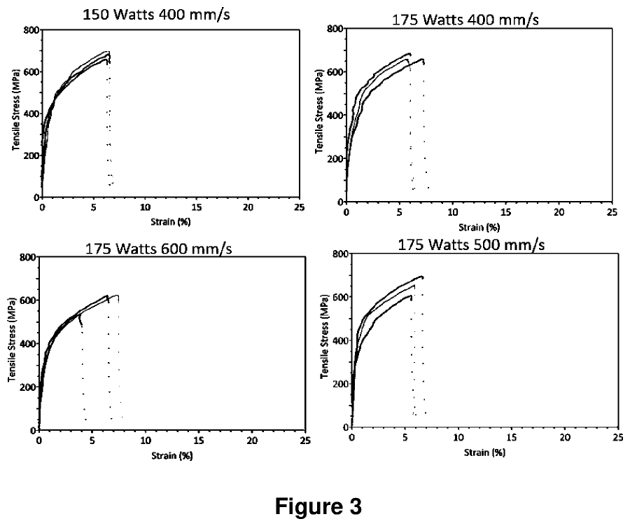

Usefully, the inventor has found that the alloy of the present invention, is biodegradable. Surprisingly, the inventor has also found that the alloy of the present invention has a lower tendency to biodegrade, however has a higher corrosion rate once biodegradation occurs (see FIG. 3 and Example 2, below). The estimated corrosion current density of the products comprising the alloy of the present invention, compared to the alloy cast using conventional methods, may be up to 10% higher (that is, up to 110% of the estimated corrosion current density of the reference material), or up to 20% higher (that is, up to 120% of the estimated corrosion current density of the reference material), or up to 30% higher (that is, up to 130% of the estimated corrosion current density of the reference material), or up to 40% higher (that is, up to 140% of the estimated corrosion current density of the reference material), or up to 50% higher (that is, up to 150% of the estimated corrosion current density of the reference material), or up to 60% higher (that is, up to 160% of the estimated corrosion current density of the reference material), or up to 70% higher (that is, up to 170% of the estimated corrosion current density of the reference material), or up to 80% higher (that is, up to 180% of the estimated corrosion current density of the reference material), or up to 85% higher (that is, up to 185% of the estimated corrosion current density of the reference material), or up to 90% higher (that is, up to 190% of the estimated corrosion current density of the reference material), or up to 95% higher (that is, up to 195% of the estimated corrosion current density of the reference material), or up to 100% higher (that is, up to 200% of the estimated corrosion current density of the reference material), or more. It is an advantage of the present invention that the additive manufactured products comprising an alloy of the present invention displays a lower tendency to start corroding, but an increased estimated corrosion current density, compared to the reference alloy. Such corrosion characteristics are particularly suitable for biological uses, whereby corrosion is resisted initially, however once degradation starts, it is more rapidly degraded.

Mechanical Properties

As shown below in Example 3, the parameters of the additive manufacturing process can be used to tailor, or optimise, the mechanical properties of the obtained products. For instance, it is a general principal, for at least for the alloy of the present invention when used in an additive manufacturing process, the combination of increasing laser strength and increasing scan rate of the laser results in a more brittle product (that is, with a with a lower tensile strength required to break the material).

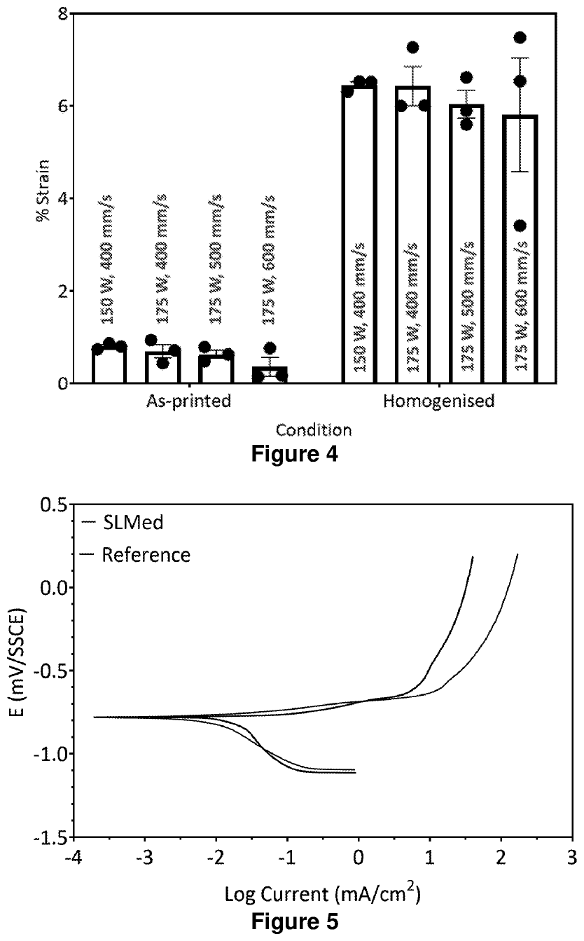

Surprisingly, it has been shown by the inventor that these mechanical differences provided by the manufacturing conditions are maintained even after homogenisation, indicating that substantial and lasting differences exist as a result of the additive manufacturing conditions (see FIGS. 4 to 6) and which are not ameliorated by heat treatment. In particular, as seen in FIG. 4, although homogenization increased the % strain at which the ultimate strength was achieved compared to the “as-printed” articles, the order (from highest to lowest % strain) remained as (1) 150 W, 400 mm/s, (2) 175 W, 400 mm/s, (3) 175 W, 500 mm/s, and (4) 175 W, 600 mm/s.

It is anticipated that the skilled person could use this principal to use the alloy of the present invention to produce suitable medical devices using additive manufacturing techniques. As mentioned above, most biodegradable materials used in biological applications are based on thermoplastic polymers. However, such polymers generally have a low intrinsic strength, and so are not suitable for uses that require some strength (such is in load bearing applications, or for some cardiovascular uses, such as arterial stents). Although alloys appear to be a suitable replacement, there are generally stiffer materials (i.e., have a higher modulus of elasticity). If used with a material with a low elastic modulus in a load bearing environment (such as, for example, as an implant to support a bone fracture during healing), severe issues can arise for the patient. Accordingly, the alloy of the present invention, in combination with additive manufacturing processes, may be tuned to reduce the elastic properties of the material by altering the manufacturing parameters and post-processing treatments,

Strain Recovery

The inventor has also found that the strain recovery achieved by products comprising the alloy of the present invention and produced using additive manufacturing techniques and then homogenized are comparable to shape memory alloys produced with conventional techniques. In other words, as shown below in Example 4, strain recovery of up to about 4% is achievable with additive manufactured products comprising the alloy of the present invention. As the skilled person would appreciate, iron-based shape memory alloys have been shown to have up to about 7-9% strain recovery. Further, the inventor expects that improvements in strain recovery could be achieved by optimisation of the parameters used during additive manufacture, as well as the use of various post-processing techniques. Put differently, although Example 4 shows strain recovery of up to about 4%, this is expected to be optimisable.

Uses

The shape memory ability of the alloy of the present invention means that devices such as medical devices may be shaped into a desired geometry at certain temperatures (e.g., a temperature lower than body temperature) by external forces. After installation/implantation the devices may return to their original shape or geometry when subject to external stimuli such as heat (body temperature) or a chemical or catalyst or other external stimuli. This provides a one-way shape memory where only two geometries are involved. Two-way shape memory of these materials can provide a third geometry which the devices can be shaped into under external stimuli or otherwise.

The shape memory devices may have the ability to generate constant and lighter forces during the insertion and positioning process. This is mainly because the device may be used in its pseudoplastic state where stress is not significantly increased by strain increments and this unique property of shape memory alloys may help protect the underlaying equipment and reduce patient discomfort, along with preventing hyalinization.

Preferably, the alloy of the present invention is used in additive manufacturing processes to produce devices, particularly devices for medical applications, whereby the shape memory functions, mechanical properties and degradability can be tuned by adjusting manufacturing parameters and processes. It is also envisioned that devices for use in other areas (e.g., removable actuators, etc.) may be achieved by additive manufacturing, utilising the alloy of the present invention.

The alloys may be used to prepare articles of manufacture for use in medical applications. For example, bone screws, plates, nails, stets, tubes, orthopaedic implants, orthopaedic braces, scaffolds, spinal cages and the like may all be manufactured using the alloy of the present invention, particularly with additive manufacturing processes as discussed herein.

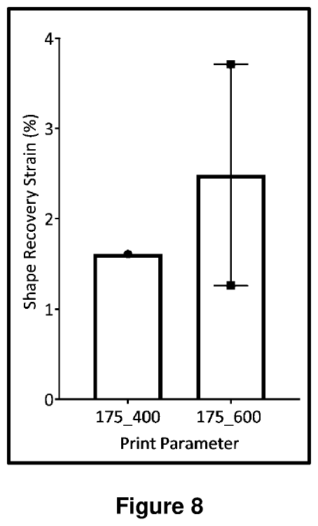

By way of illustrative example, FIG. 7 shows biodegradation and bone healing in a printed shape memory article. The material stability of a product is indicated on the vertical axis, while the time is indicated on the horizontal axis. Bone support provided by the shape memory alloy decreases in material stability over the course of months to years. The graph illustrates the complementary increase in stability of bone as recovery occurs. This means a consistent bone support over years. In contrast a permanent implant does not decrease in stability.

### EXAMPLES

The inventor of the present invention has characterised the alloy described herein. The following exemplary embodiments serve only to highlight features of the present invention, they are not intended to limit the invention as defined by the claims.

In the following examples, the additive manufacturing processes were performed using a laser powder bed fusion process on a commercial machine. The parameters and their limits for this machine are: Laser power: 30 W to 1 kW; Scan speed: 30 mm/s up to 10,000 mm/s; Hatch spacing: 20 μm up to 200 μm; scan strategy: unidirectional scanning, bidirectional zig-zag scanning, chessboard scanning, random scanning, re-scanning of the same layer, rotation between adjacent layer with 90 degree or at 67 degree or other angles; layer thickness: 5 um up to 150 um; and beam shape: Gaussian like beam shape, or top hat.

The parameters that were kept constant for the below examples were: Hatch spacing: 45 μm; ad Layer thickness of 30 μm. The scanning strategy was selected from bidirectional zig-zag scanning and re-scanning, however the inventor does not consider that the choice of scanning strategy resulted in any material variation in the articles produced.

### Example 1—Composition

In this example, a mixed feed powder is composed of Fe, Mn, and Si, and has a nominal composition of 64 wt. %, 30 wt. %, and 6 wt. %, respectively. Identical articles were then produced from this powder mixture using a laser-PBF process, with the laser power and scanning speed varied. An EDS analysis was used to roughly estimate the resulting contribution of the elements in the alloy. The composition of the elements at different laser powers and scan speeds is presented in FIG. 2.

As can be seen from this figure, the results show that the composition (in terms of relative proportions of Fe, Mn and Si) can vary depending on the parameters. Notably, Mn and, to a lesser degree, Si, are evaporated during manufacture to a greater degree than Fe, resulting in the relative decrease in Mn and Si in the produced alloy articles, and a relative increase in Fe in the produced alloy. Further, this effect appears to correlate with increasing laser power, as an even greater change in concentration was generally seen when the laser power was at 175 W, compared to 150 W.

### Example 2—Biodegradation

In vitro corrosion tests have also been carried out on the reference alloy (that is, the alloy of the present invention that has not been produced by additive manufacturing) and an article that has been fabricated using an additive manufacturing technique (in particular, a laser-PBF process).

The corrosion susceptibility of the reference alloy and the 3D printed alloy were compared through an in vitro corrosion electrochemical analysis. The test was carried out in a Hanks' balanced salt solution at 37±2° C., and the results are presented in FIG. 3. As the skilled person would appreciate, a Hank's balanced salt solution (usually comprising sodium chloride, potassium chloride, calcium chloride, magnesium sulfate heptahydrate, magnesium chloride hexahydrate, sodium phosphate dibasic dihydrate, glucose and sodium bicarbonate) is generally used as a buffer system in cell culture media and is at physiological pH and osmotic pressure, making this salt system appropriate to test the biodegradability of materials.

Following exposure of the alloy materials to the Hanks' balanced salt solution, Tafel extrapolation was performed on the gathered data to estimate the corrosion potential, E_{corr} (mV), and the corrosion current density, I_{corr} (μA/cm³). As the skilled person would be aware, Tafel extrapolation is commonly used to estimate the corrosion behaviour of a material, whereby when the log of the current is plotted against the energy, the corrosion potential is the extrapolated y-intercept, and the corrosion current density is the extrapolated mid-point between the linear portion of the anodic slope and the linear portion of the cathodic slope. The corrosion parameters derived from FIG. 3 are summarised in Table 1 below.

TABLE 1
The corrosion parameters derived from
the Tafel extrapolation of FIG. 3.

| | E_{corr} (mV) | I_{corr} (uA/cm²) |
|---|---|---|
| Additive manufactured alloy | −777.66 | 8.50 |
| Reference alloy | −846.60 | 1.53 |

The E_{corr} represents a thermodynamic parameter that corresponds to the corrosion tendency of the alloy. It can be observed that the additive manufactured alloy has a lower E_{corr} value suggesting a more stable material than the reference alloy. However, it is the kinetic parameter, loom that is necessary in predicting the corrosion rate as it is directly proportional to the rate of corrosion. The estimated corrosion current density of the additive manufactured alloy is over 80% higher than the reference alloy. This suggests that the additive manufactured alloy will undergo a faster degradation than the reference alloy.

### Example 3—Mechanical Properties

Articles of a standard shape comprising an alloy with a nominal composition of 64 wt. % Fe, 30 wt. % Mn, and 6 wt. % Si were produced using a laser-PBF process and tested for various mechanical properties. Samples were produced at different laser power and scanning speeds, with all other parameters maintained at constant values.

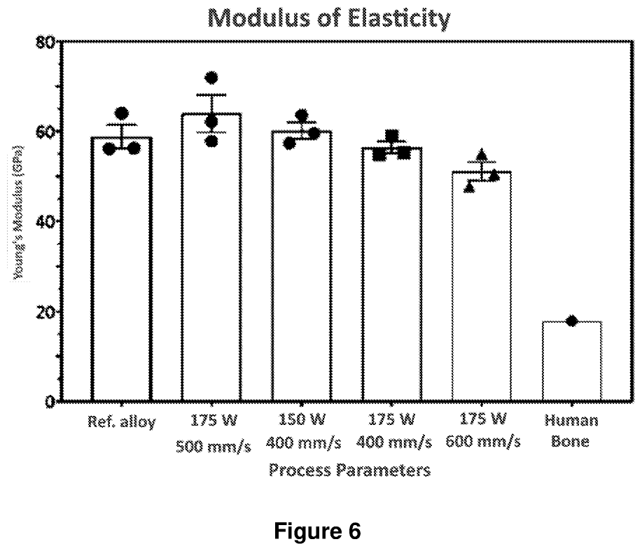

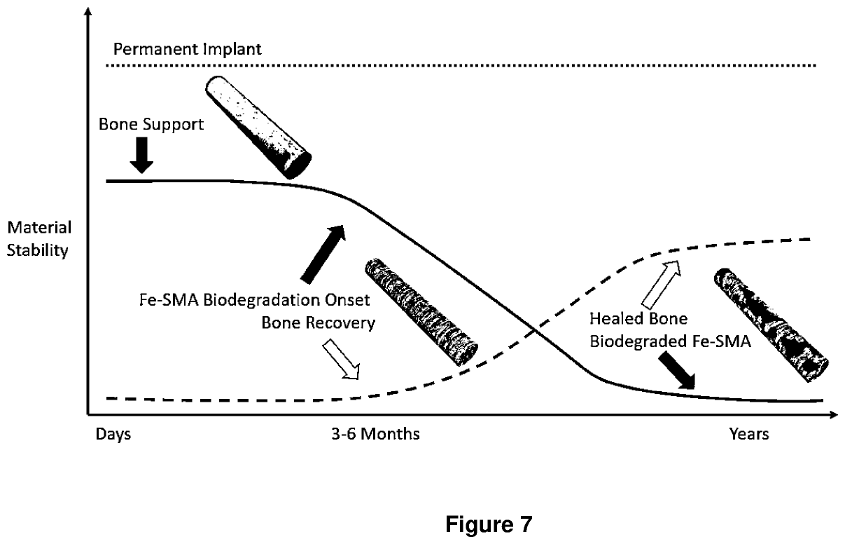

In a first test, tensile strength and ductility of the as-fabricated samples (without post processing) and after post-processing homogenization were conducted on a standard Instron testing apparatus, with three samples tested per set of manufacturing parameters and post-processing treatment. A graph of the % strain at the ultimate strength is shown in FIG. 4 (whereby the ultimate strength is the highest point on a stress-strain plot). The stress-strain curves for the homogenized samples are shown in FIG. 5 and the resulting modulus of elasticity for these samples is shown in FIG. 6.

These figures show that the elasticity of the samples generally declines as laser power and scanning speed increases, and the samples become more variable. This is likely because more defects are introduced by the laser-PBF process at higher laser powers and scanning speeds. Interestingly, this impact on the elasticity of the material is maintained even after a post-processing heat treatment, indicating that the alloys of the present invention can “remember” the manufacturing parameters, at least in regard to the laser power and scanning speed.

### Example 4—Strain Recovery

In order to test the shape memory ability of the alloy of the present invention following an additive manufacturing process, the strain recovery was examined, The free strain recovery of articles fabricated using a laser-PBF process, followed by post-processing homogenisation, were tested and compared to a reference alloy in order to assess the shape memory performance of the fabricated alloy. The alloy used in the laser-PBF process and as the reference alloy are the same as used in the above examples.

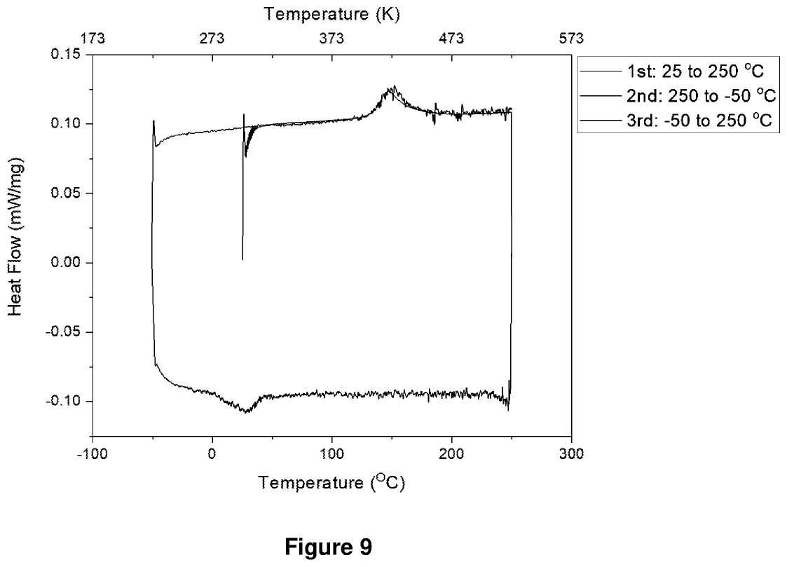

The free strain recovery tests were conducted at room temperature, where a 4% strain was applied to the specimen using a standard Instron apparatus at a rate of 1.67 E⁻⁴ mm/s. After the desired strain level was reached, the load was removed at a similar strain rate and the samples were then heated for 10 minutes in a 600° C. furnace with argon cover. The results shown in FIG. 8 show the strain recovery for two samples fabricated using a laser-PBF process, at different scanning speed (44 mm/s vs 600 mm/s). All other parameters remained constant, including the laser power (which is 175 W). As can be seen, the strain recovery is affected by the manufacturing parameters and can be enhanced by using a faster scanning speed.

### Example 5—Phase Transformation

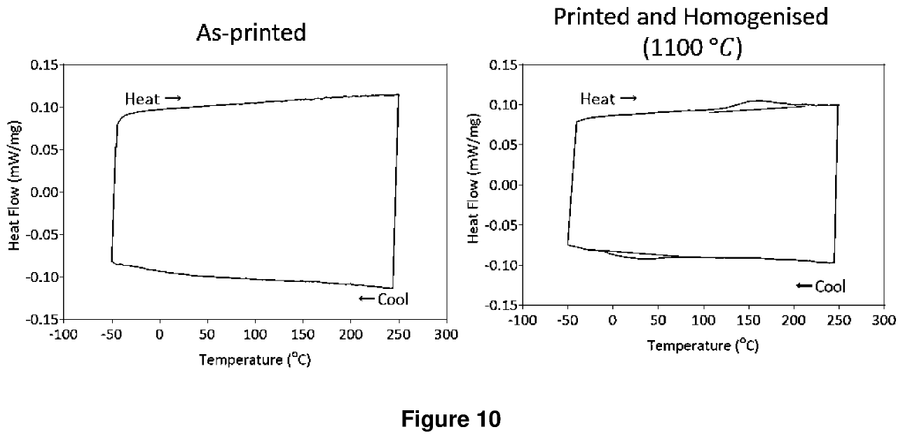

The critical phase transformation characteristic temperatures for the additive manufactured shape memory alloys described above was tested and is shown in FIG. 9. The shape memory properties, and the necessary external stimuli: temperature and force of the fabricated shape memory alloy may be tailored and controlled through alloy composition control and additive manufacturing process control. This capability means that the shape memory alloys parts fabricated may be tailored to meet different properties requirements in different industrial fields, covering broad market needs.

Additionally, post-processing steps can also act to improve the phase transformation characteristics of alloys of the present invention, when fabricated using additive manufacturing techniques. As shown in FIG. 10, there are shallow phase changes between about 130° C. and about 180° C. on the heat phase, and between 50° C. and 0° C. on the cool phase, for the “as fabricated” (i.e., “as printed”) sample. However, these heat flow changes are more pronounced after a homogenization (i.e., annealing) step, carried out at 1100° C. Whilst the skilled person would consider both products to be shape memory alloy products, this effect is more pronounced in the post-processed sample.

### Forms of the Invention

Forms of the present invention include:
1. A biodegradable shape memory alloy, the alloy comprising a metallic alloy.
2. A biodegradable shape memory alloy comprising Fe, Mn and Si.
3. The biodegradable shape memory alloy of form 1 or form 2, comprising:
   Mn at between 5 and 75% by weight;
   Si at between 1 and 15% by weight; and
   the balance being Fe.
4. The biodegradable shape memory alloy of any one of forms 1 to 3, comprising:
   Mn at between 5 and 50 wt %, or between 20 and 30% by weight;
   Si at between 1 and 15% by weight, or between 3 and 9% by weight; and
   the balance being Fe.
5. The biodegradable shape memory alloy of any one of forms 1-4, consisting of:
   Mn at between 28 and 30% by weight;
   Si at between 5 and 7% by weight; and
   Fe at between 63 and 67% by weight.
6. A biodegradable shape memory alloy, consisting of:
   Mn at 30% by weight;
   Si at 6% by weight; and
   Fe at 64% by weight.
7. A biodegradable shape memory alloy as defined in form 1, wherein the metallic alloy is composed of any of a group of alloys including Fe— Pt, Fe—Pd, Fe—Cu, Fe—Co, Fe—Cr, Fe—Mn, and Fe—Ni.
8. A biodegradable shape memory alloy as defined in any one of forms 2 to 7, further composed of an additional element.
9. The biodegradable shape memory alloy of any one of forms 1 to 8, further comprising at least one additional element selected from Cr, Ni, Ag, Nb, Ti, V, Co, Al, W, Sn, B, N, C, S and rare earth elements.
10. An article composed of the biodegradable shape memory alloy of any of the preceding forms.
11. An article as defined in form 10, wherein the article comprises an implantable medical device.
12. An article as defined in form 10 or 11, the article being manufactured through additive manufacturing.
13. A material for additive manufacturing, the material comprising:
   Mn at between 25 and 35% by weight;
   Si at between 5 and 8% by weight;
   Fe at between 60 and 70% by weight, and
   optionally comprising at least one additional element selected from Cr, Ni, Ag, Nb, Ti, V, Co, Al, W, Sn, B, N, C, S and rare earth elements.
14. The material of form 13, wherein the material is a powder.
15. The material of form 13 or form 14, wherein the powder is blended from elemental powders, or is prealloyed and then ground to form a powder.
16. A process for manufacturing an article comprising an alloy of any one of forms 1-9, the process comprising:
   obtaining the material of any one of forms 13 to 15 for use in an additive manufacturing process; and
   carrying out the additive manufacturing process to obtain the article, wherein the article recovers from strain with heating.
17. The process of form 16, wherein the recovered strain is about 10% or about 8% or about 4%.
18. The process of form 17, wherein the strain is recovered upon heating.
19. The process of form 18, wherein the heating comprises heating the article to least 30° C., at least 50° C., at least 70° C., at least 400° C., or at least 600° C.
20. The process of any one of forms 16 to 19, wherein the additive manufacturing process is a laser powder bed fusion (PBF) process, or a selective laser melting (SLM) process.
21. The process of any one of forms 16 to 20, wherein the article is a medical device.
22. The process of form 21, wherein the article is an implantable medical device.
23. The process of form 22, wherein the article is a bone implant.
24. The process of any one of forms 16 to 23, further comprising a homogenization step.
25. A process for tuning elemental composition of an alloy, comprising the steps:
   obtaining a material for use in additive manufacturing, the material comprising at least two elements; and
   exposing the material to an additive manufacturing process;
   whereby, in use, at least a portion of at least one element of the alloy is evaporated during the additive manufacturing process.
26. The process of form 25, wherein the at least one element of the alloy that is evaporated has the lowest melting point temperature.
27. The process of form 25 or form 26, wherein the material is as defined in any one of forms 13 to 15.
28. The process of form 27, wherein the element that is at least partially evaporated is Mn and/or Si.
29. The process of any one of forms 25 to 28, wherein the evaporation is controlled by adjusting parameters of the additive manufacturing process.
30. The product produced by the process of any one of forms 16 to 29.

Although the invention has been described with reference to specific examples, it will be appreciated by those skilled in the art that the invention may be embodied in many other forms in particular features of any one of the various described examples may be provided in any combination in any of the other described examples. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

1. A biodegradable shape memory alloy comprising or consisting of Fe, Mn and Si.

2. The biodegradable shape memory alloy of claim 1, comprising or consisting of:
Mn at between 5 and 75% by weight;
Si at between 1 and 15% by weight; and
the balance being Fe.

3. The biodegradable shape memory alloy of claim 1 or claim 2, comprising or consisting of:
Mn at between 20 and 30% by weight;
Si at between 3 and 9% by weight; and
the balance being Fe.

4. The biodegradable shape memory alloy of any one of claims 1-3, comprising or consisting of:
Mn at between 28 and 30% by weight;
Si at between 5 and 7% by weight; and
Fe at between 63 and 67% by weight.

5. A biodegradable shape memory alloy, comprising or consisting of:
Mn at 30% by weight;
Si at 6% by weight; and
Fe at 64% by weight.

6. The biodegradable shape memory alloy of any one of claims 1-3, further comprising at least one additional element selected from Cr, Ni, Ag, Nb, Ti, V, Co, Al, W, Sn, B, N, C, S and rare earth elements.

7. A material for additive manufacturing, the material comprising:
Mn at between 25 and 35% by weight;
Si at between 5 and 8% by weight;
Fe at between 60 and 70% by weight, and
optionally comprising at least one additional element selected from Cr, Ni, Ag, Nb, Ti, V, Co, Al, W, Sn, B, N, C, S and rare earth elements.

8. The material of claim 7, wherein the material is a powder.

9. The material of claim 7 or claim 8, wherein the powder is blended from elemental powders, or is prealloyed and then ground to form a powder.

10. A process for manufacturing an article comprising an alloy of any one of claims 1-6, the process comprising:
obtaining the material of any one of claims 7 to 9 for use in an additive manufacturing process; and
carrying out the additive manufacturing process to obtain the article, wherein the article recovers from strain with heating.

11. The process of claim 10, wherein the recovered strain is about 10% or about 8% or about 4%.

12. The process of claim 11, wherein the strain is recovered upon heating.

13. The process of claim 12, wherein the heating comprises heating the article to least 30° C., at least 50° C., at least 70° C., at least 400° C., or at least 600° C.

14. The process of any one of claims 10 to 13, wherein the additive manufacturing process is a laser powder bed fusion (PBF) process, or a selective laser melting (SLM) process.

15. The process of any one of claims 10 to 14, wherein the article is a medical device.

16. The process of claim 15, wherein the article is an implantable medical device.

17. The process of claim 16, wherein the article is a bone implant.

18. The process of any one of claims 10 to 17, further comprising a homogenization step.

19. A process for tuning elemental composition of an alloy, comprising the steps:
obtaining a material for use in additive manufacturing, the material comprising at least two elements; and
exposing the material to an additive manufacturing process;
whereby, in use, at least a portion of at least one element of the alloy is evaporated during the additive manufacturing process.

20. The process of claim 19, wherein the at least one element of the alloy that is evaporated has the lowest melting point temperature.

21. The process of claim 19 or claim 20, wherein the material is as defined in any one of claims 7 to 9.

22. The process of claim 21, wherein the element that is at least partially evaporated is Mn and/or Si.

23. The process of any one of claims 19 to 22, wherein the evaporation is controlled by adjusting parameters of the additive manufacturing process.

24. The product produced by the process of any one of claims 10 to 23.
